(19) United States
(12) Patent Application Publication
(10) Pub. No.: US 2024/0351964 A1
(43) Pub. Date: Oct. 24, 2024

(54) CRACKER RECYCLES TO VALUE ADDED CHEMICALS

(71) Applicant: SABIC Global Technologies B.V., Bergen op Zoom (NL)

(72) Inventors: Shehzada Khurram, Bangalore (IN); Ernesto Uehara, Bangalore (IN); Chandrasekar Subramani, Jubail (SA); Rathinam Jothi Mahalingam, Jubail (SA)

(21) Appl. No.: 18/685,628

(22) PCT Filed: Sep. 12, 2022
(86) PCT No.: PCT/EP2022/075203

§ 371 Date: Feb. 22, 2024

(30) Foreign Application Priority Data
Sep. 14, 2021 (EP) 21196526.4

(51) Int. Cl. C07C 7/00; C07C 5/27; C07C 7/04; C07C 7/163; C07C 9/18; C07C 11/10

(52) U.S. Cl. CPC C07C 7/005; C07C 5/2708; C07C 7/04; C07C 7/163; C07C 9/18; C07C 11/10

(57) ABSTRACT

A process for producing isopentane and isoamylene can comprise passing a feed stream comprising C5 hydrocarbons through a hydrodesulfurization assembly to produce a first stream: passing the first stream to a first separation assembly to produce a second stream and a first separated stream: passing the second stream to a deisopentanizer assembly to produce a second separated stream comprising isopentane, a third separated stream comprising C6+ hydrocarbons, and a raffinate stream comprising C5 hydrocarbons; and passing the raffinate stream to a reactor assembly to produce a reaction product stream comprising isoamylene. The present disclosure also provides for a system for producing isopentane and isoamylene.

### BACKGROUND

Hydrocarbon cracking processes are important conversion processes used in petroleum refineries. Recycle streams of crude hydrocarbons are often formed as byproducts during these cracking processes.

In general, it is noted that C5 hydrocarbons can serve a number of very different businesses (e.g., commodity elastomers, tackifiers, high performance plastics, specialty chemicals, fine and performance chemicals, etc.). Such a value chain can generally be described at a high level as: isoprene, dicyclopentadiene, piperylene and isoamylene.

It is noted that streams comprising C5 hydrocarbons (e.g., a C5 rich stream coming from a gasoline hydrogenation unit (GHU), or a separated C5 stream before a GHU and then hydrogenated) are generally sent back to the cracker furnaces for further cracking.

### BRIEF DESCRIPTION

As described above, conventional practice provides that streams comprising C5 hydrocarbons (e.g., a C5 rich stream coming from a gasoline hydrogenation unit (GHU), or a separated C5 stream before a GHU and then hydrogenated) are simply sent back to the cracker furnaces for further cracking. However, C5 hydrocarbons can serve a number of very different businesses (e.g., commodity elastomers, tackifiers, high performance plastics, specialty chemicals, fine and performance chemicals, etc.). A solution to address the deficiencies of conventional processes has been discovered. The disclosed process is premised on advantageously utilizing at least a portion of a stream (e.g., hydrogenated stream) comprising C5 hydrocarbons for isopentane and isoamylene production. It is noted that as used herein, isoamylene comprises at least one of 2-methyl-2-butene, or 3-methyl-1-butene, or combinations thereof. The discovered process thereby enables commercially valuable isopentane and isoamylene production from a stream (e.g., hydrogenated stream) comprising C5 hydrocarbons, instead of simply sending such a stream back to the cracker furnaces for further cracking.

These and other inefficiencies and opportunities for improvement are addressed or overcome by the processes, systems and methods of the present disclosure.

The present disclosure provides advantageous processes, systems and methods for utilizing at least a portion of a stream (e.g., hydrogenated stream) comprising C5 hydrocarbons for isopentane and isoamylene production.

The present disclosure provides for a process for producing isopentane and isoamylene comprising passing a feed stream comprising C5 hydrocarbons through a hydrodesulfurization assembly to produce a first stream; passing the first stream to a first separation assembly to produce a second stream and a first separated stream; passing the second stream to a deisopentanizer assembly to produce a second separated stream comprising isopentane, a third separated stream comprising C6+ hydrocarbons, and a raffinate stream comprising C5 hydrocarbons; and passing the raffinate stream to a reactor assembly to produce a reaction product stream comprising isoamylene.

The present disclosure also provides for a system for producing isopentane and isoamylene comprising a hydrodesulfurization assembly to produce a first stream from a feed stream comprising C5 hydrocarbons; a first separation assembly to separate the first stream and produce a second stream and a first separated stream; a deisopentanizer assembly to separate the second stream and produce a second separated stream comprising isopentane, a third separated stream comprising C6+ hydrocarbons, and a raffinate stream comprising C5 hydrocarbons; and a reactor assembly to produce a reaction product stream comprising isoamylene from the raffinate stream.

The above described and other features are exemplified by the following FIGURES and detailed description.

Any combination or permutation of embodiments is envisioned. Additional advantageous features, functions and applications of the disclosed processes, systems and methods of the present disclosure will be apparent from the description which follows, particularly when read in conjunction with the appended FIGURES. All references listed in this disclosure are hereby incorporated by reference in their entireties.

### BRIEF DESCRIPTION OF THE DRAWINGS

The following FIGURES are exemplary embodiments wherein the like elements are numbered alike.

FIG. 1 is a schematic of a system and process for producing isopentane and isoamylene, according to the present disclosure.

### DETAILED DESCRIPTION

The exemplary embodiments disclosed herein are illustrative of advantageous systems for producing isopentane and isoamylene, and processes of the present disclosure and methods/techniques thereof. It should be understood, however, that the disclosed embodiments are merely exemplary of the present disclosure, which may be embodied in various forms. Therefore, details disclosed herein with reference to exemplary systems for producing isopentane and isoamylene and associated processes/techniques of use are not to be interpreted as limiting, but merely as the basis for teaching one skilled in the art how to make and use the advantageous systems for producing isopentane and isoamylene of the present disclosure.

The present disclosure provides advantageous processes, systems and methods for utilizing at least a portion of a stream (e.g., hydrogenated stream) comprising C5 hydrocarbons for isopentane and isoamylene production.

A more complete understanding of the components, processes, systems and apparatuses disclosed herein can be obtained by reference to the accompanying drawings. These FIGURES (also referred to herein as “FIG.”) are merely schematic representations based on convenience and the ease of demonstrating the present disclosure, and are, therefore, not intended to indicate relative size and dimensions of the devices or components thereof and/or to define or limit the scope of the exemplary embodiments. Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

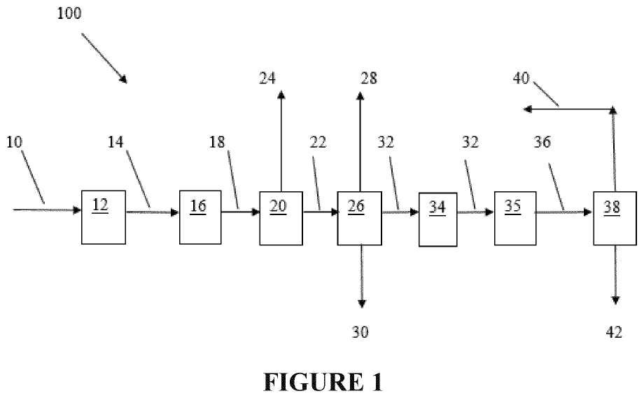

FIG. 1 is a schematic of a system and process 100 for producing isopentane and isoamylene, according to the present disclosure. With reference to FIG. 1, a feedstock stream 10 (e.g., hydrocarbon feedstock stream 10) can be fed to a cracker furnace assembly 12.

In general, hydrocarbon cracking processes 12 are important conversion processes used in petroleum refineries. For example, fluid catalytic cracking (FCC) is widely used to convert the high-boiling, high-molecular weight hydrocarbon fractions of petroleum crude oils to more valuable gasoline, olefinic gases, and other products. Thermal cracking of naphtha and gas oil is also widely used in the petrochemical industry to produce a variety of olefins and aromatics. For example, hydrocarbon feedstock streams 10 can be mixed with steam and subjected to elevated temperatures (e.g., 700 to 900° C.) in a steam cracker furnace assembly 12 wherein the feedstock components are cracked into various fractions. The effluent of the steam cracker 12 can contain a gaseous mixture of hydrocarbons, for example, saturated and unsaturated olefins and aromatics (C1-C35). The effluent can then be separated into individual olefins (for example, ethylene, propylene, C4's, C5's) and a pyrolysis gasoline (“pygas”) stream. Recycle streams of crude hydrocarbons are often formed as byproducts during these cracking processes 12.

For example, a feed stream 14 comprising C5 hydrocarbons can be produced by cracker furnace assembly 12. After the cracker furnace assembly 12, feed stream 14 comprising C5 hydrocarbons can be hydrogenated (e.g., feed stream 14 can be a C5 rich stream coming from a gasoline hydrogenation unit (GHU); or feed stream 14 can be a separated C5 stream before a GHU and then hydrogenated). Current practice provides that such hydrogenated feed streams 14 are generally sent back to the cracker furnaces 12 for further cracking.

The present disclosure advantageously provides that at least a portion of feed stream 14 (e.g., hydrogenated feed stream 14) comprising C5 hydrocarbons can be utilized for isopentane and isoamylene production.

The feed stream 14 (e.g., hydrogenated feed stream 14) can comprise 0 to 2 weight percent (“wt %”) C4 hydrocarbons, 10 to 20 wt % isopentane, 15 to 30 wt % pentane, 10 to 20 wt % pentene, 0 to 1 wt % isoamylene, 10 to 30 wt % other C5 hydrocarbons (C5 hydrocarbons excluding isopentane, pentane, pentene and isoamylene), and 15 to 30 wt % C6+ hydrocarbons, based on a total weight of the feed stream 14. For example, the other C5 hydrocarbons of feed stream 14 (C5 hydrocarbons excluding isopentane, pentane, pentene and isoamylene) can comprise cyclopentadiene, cyclopentane and cyclopentene.

It is noted that the feed stream 14 can have a boiling point ranging from 30° C. to 36° C., although the present disclosure is not limited thereto.

For example and as shown in FIG. 1, at least a portion of stream 14 (e.g., hydrogenated stream 14) comprising C5 hydrocarbons can be fed to a hydrodesulfurization assembly/reactor 16. In general, the hydrodesulfurization assembly 16 is configured and adapted to treat stream 14 comprising C5 hydrocarbons to reduce the sulfur content of stream 14, thereby producing first process stream 18 with reduced sulfur content relative to stream 14. For example, stream 14 can comprise 20 to 50 parts per million by weight (ppm) sulfur prior to introduction to the hydrodesulfurization assembly 16, and first process stream 18 can comprise less than 1 ppm sulfur after treatment via hydrodesulfurization assembly 16.

The first process stream 18 can then be fed to a separation/stripper assembly 20 to produce a second process stream 22 comprising C5 hydrocarbons, and to produce a first separated stream 24 comprising H₂S and C4 hydrocarbons. In a non-limiting example, the separation/stripper assembly 20 can be operated at a top temperature of 35 to 80° C. and at a bottom temperature of 140 to 165° C., and at a pressure of 5 to 8 barg (500 to 800 kilopascals).

The second process stream 22 exiting the separation/stripper assembly 20 can comprise 70 to 80 wt % C5 hydrocarbons, 0 to 0.50 wt % C4 hydrocarbons, 0 to 0.10 wt % H₂S, and 15 to 30 wt % C6 hydrocarbons, based on a total weight of the second process stream 22.

The first separated stream 24 (e.g., exiting from the top of the separation/stripper assembly 20) can comprise 0 to 0.01 wt % H₂S, and 0 to 2 wt % C4 hydrocarbons, based on a total weight of the first separated stream 24 (e.g., exiting from the top of the separation/stripper assembly 20).

The second process stream 22 can be fed to a deisopentanizer assembly 26 to produce a second separated stream 28, a third separated stream 30, and a raffinate stream 32 comprising C5 hydrocarbons. In exemplary embodiments, the second separated stream 28 comprises isopentane, and the third separated stream 30 comprises C6+ hydrocarbons. In a non-limiting example, the deisopentanizer assembly 26 can be operated at 70 to 88 stages, and at a top temperature of 50 to 60° C. and at a bottom temperature of 70 to 85° C., and at a pressure of 2 to 3.1 barg (200 to 310 kilopascals).

It is noted that the raffinate stream 32 comprising C5 hydrocarbons can be withdrawn from a side of the deisopentanizer assembly 26 (e.g., raffinate stream 32 is a side draw from assembly 26).

The second separated stream 28 can comprise 68 to 75 wt % isopentane, 5 to 10 wt % n-pentane, and 15 to 25 wt % 1-pentene, based on a total weight of the second separated stream 28.

The third separated stream 30 can comprise 15 to 28 wt % C6+ hydrocarbons, and 0.18 to 0.30 wt % dicyclopentadiene, based on a total weight of the third separated stream 30.

Some advantages of utilizing the deisopentanizer assembly 26 (where second separated stream 28 comprising isopentane and third separated stream 30 comprising C6+ hydrocarbons are separated) before sending the raffinate stream 32 comprising C5 hydrocarbons for further processing to produce isoamylene can include: (i) providing isopentane as a product from the deisopentanizer assembly 26 (e.g., via separated stream 28 comprising isopentane), and the isopentane product can be further utilized to produce polyethylene or polystyrene (e.g., via a polyethylene plant), and also by decreasing the isopentane in the eventual recycle stream 40 (e.g., about 30% less isopentane in stream 40 relative to conventional processes), this can thereby enhance the cracker 12 run length; and/or (ii) in the third separated stream 30 comprising C6+ hydrocarbons, mostly benzene can be recovered, which can reduce the coke formation in cracker 12 (e.g., via eventual recycle stream 40, which now has less C6+ hydrocarbons relative to conventional processes).

The raffinate stream 32 can comprise 1 to 5 wt % isopentane, 0 to 1 wt % isoamylene, 90 to 98 wt % other C5 hydrocarbons (C5 hydrocarbons excluding isopentane and isoamylene), and 0.10 to 1 wt % C6+ hydrocarbons, based on a total weight of the raffinate stream 32.

For example, the other C5 hydrocarbons of raffinate stream 32 (C5 hydrocarbons excluding isopentane and isoamylene) can comprise cyclopentadiene, cyclopentane and cyclopentene.

It is noted that the raffinate stream 32 can have a boiling point ranging from 30° C. to 40° C., although the present disclosure is not limited thereto.

The raffinate stream 32 can comprise dienes or diolefins in an amount of less than or equal to 5 weight %, such as less than or equal to 1 weight %, based on a total weight of the raffinate stream 32.

The raffinate stream 32 comprising C5 hydrocarbons can be fed to a reactor assembly 35 to produce a reaction product stream 36 comprising isoamylene. In some embodiments and as shown in FIG. 1, the raffinate stream 32 can be heated via a heater assembly 34 (e.g., to a temperature of 70 to 160° C.) prior to introduction to the reactor assembly 35.

The reaction product stream 36 can comprise 55 to 63 wt % isoamylene, 2 to 3 wt % C6+ hydrocarbons, and 35 to 43 wt % other C5 hydrocarbons (C5 hydrocarbons excluding isoamylene), based on a total weight of the reaction product stream 36.

In general, passing or feeding the raffinate stream 32 comprising C5 hydrocarbons to the reactor assembly 35 to produce the reaction product stream 36 can comprise contacting the raffinate stream 32 with a catalyst (e.g., a zeolite-based catalyst) to produce the reaction product stream 36.

For example, it is noted that the raffinate stream 32 can be contacted with the catalyst in reactor assembly 35 at a temperature of 100 to 210° C., and at a pressure of 10 to 30 barg (1,000 to 3,000 kilopascals) (e.g., under enough pressure to keep the reactants in liquid phase) to produce the reaction product stream 36.

In general, the raffinate stream 32 comprises pentene, and the reactor assembly 35 is configured and adapted to convert at least a portion of the pentene present in the raffinate stream 32 to isoamylene. For example, reactor assembly 35 can be configured and adapted to convert greater than or equal to 58 weight % (e.g., 58 to 65 wt %) of the pentene present in the raffinate stream 32 to isoamylene, based on a total weight of the pentene in the raffinate stream 32. It is noted that as used herein, pentene comprises at least one of 1-pentene, or 2-pentene, or combinations thereof.

The reaction product stream 36 can be fed to a separation assembly 38 to produce: (i) a recycle stream 40 comprising C5 hydrocarbons, and (ii) a product stream 42 comprising isoamylene. In a non-limiting example, the separation assembly 38 can be operated at a temperature of 100 to 195° C., and at a pressure of 0.70 to 2.0 barg (70 to 200 kilopascals).

The recycle stream 40 can comprise 98 to 99.9 wt % C5 hydrocarbons, such as 99.91 to 99.98 wt % C5 hydrocarbons, based on a total weight of the recycle stream 40. For example, the recycle stream 40 can comprise 40 to 50 wt % pentane, 5 to 10 wt % cyclopentadiene, 10 to 15 wt % cyclopentane, 15 to 20 wt % cyclopentene, 1 to 16 wt % isopentane, and 5 to 15 wt % pentene, based on a total weight of the recycle stream 40.

In exemplary embodiments, the recycle stream 40 comprises isopentane in an amount of less than or equal to 16 weight %, such as less than or equal to 10 weight %, based on the total weight of the recycle stream 40.

It is noted that recycle stream 40 comprising C5 hydrocarbons can be recycled or fed back to at least one cracker furnace assembly 12 (e.g., at least one cracker furnace assembly 12 of system/process 100).

The product stream 42 can comprise 98 to 99.9 wt % isoamylene, based on a total weight of the product stream 42.

It is noted that the product stream 42 can have a boiling point ranging from 42° C. to 46° C., although the present disclosure is not limited thereto.

In exemplary embodiments, the resultant products of utilizing the system and process 100 for producing isopentane and isoamylene according to the present disclosure can provide the following (based on a total weight of the feed stream 14): 7 to 16 wt % of a second separated stream 28 comprising isopentane; 15 to 30 wt % of a third separated stream 30 comprising C6+ hydrocarbons; 45 to 60 wt % of a recycle stream 40 comprising C5 hydrocarbons; and 8 to 12 wt % of a product stream 42 comprising isoamylene, based on a total weight of the feed stream 14.

### Example

The following examples are merely illustrative of the processes and systems for producing isopentane and isoamylene disclosed herein and are not intended to limit the scope hereof.

An Aspen Simulation model (Aspen version-10 Simulation) was developed to predict the separation of the different streams (streams 24, 28, 30, 40 and 42) utilizing the system and process 100 for producing isopentane and isoamylene according to the present disclosure (based on a total weight of the feed stream 14): 0.30 wt % of a first separated stream 24 comprising H₂S and C4 hydrocarbons; 11.2 wt % of a second separated stream 28 comprising isopentane; 25.9 wt % of a third separated stream 30 comprising C6+ hydrocarbons; 52.9 wt % of a recycle stream 40 comprising C5 hydrocarbons; and 9.6 wt % of a product stream 42 comprising isoamylene, based on a total weight of the feed stream 14.

This disclosure further encompasses the following aspects.
Aspect 1. A process for producing isopentane and isoamylene comprising passing a feed stream comprising C5 hydrocarbons through a hydrodesulfurization assembly to produce a first stream; passing the first stream to a first separation assembly to produce a second stream and a first separated stream; passing the second stream to a deisopentanizer assembly to produce a second separated stream comprising isopentane, a third separated stream comprising C6+ hydrocarbons, and a raffinate stream comprising C5 hydrocarbons; and passing the raffinate stream to a reactor assembly to produce a reaction product stream comprising isoamylene.
Aspect 2. The process of Aspect 1 further comprising passing the reaction product stream to a separation assembly to produce: (i) a recycle stream comprising C5 hydrocarbons, and (ii) a product stream comprising isoamylene.
Aspect 3. The process of Aspect 2 further comprising recycling the recycle stream to a cracker furnace assembly.
Aspect 4. The process according to Aspect 2 or Aspect 3, wherein the recycle stream comprises isopentane in an amount of less than or equal to 16 weight %, such as less than or equal to 10 weight %, based on a total weight of the recycle stream.
Aspect 5. The process according to any one of Aspects 2 to 4, wherein the product stream comprises 98 to 99.9 wt % isoamylene, based on a total weight of the product stream.
Aspect 6. The process of any one or more of the preceding Aspects, wherein the second separated stream comprises 68 to 75 wt % isopentane, based on a total weight of the second separated stream.
Aspect 7. The process of any one or more of the preceding Aspects, wherein the deisopentanizer assembly is operated at 70 to 88 stages, and at a top temperature of 50 to 60° C. and at a bottom temperature of 70 to 85° C., and at a pressure of 2 to 3.1 barg (200 to 310 kilopascals).
Aspect 8. The process of any one or more of the preceding Aspects further comprising forming the feed stream from a portion of an effluent from a cracker furnace assembly.
Aspect 9. The process of any one or more of the preceding Aspects, wherein the raffinate stream is heated via a heater assembly to a temperature of 70 to 160° C. prior to passing to the reactor assembly.
Aspect 10. The process of any one or more of the preceding Aspects, wherein prior to passing the feed stream comprising C5 hydrocarbons through the hydrodesulfurization assembly, the feed stream is hydrogenated; and wherein the first separated stream comprises H₂S and C4 hydrocarbons.
Aspect 11. The process of any one or more of the preceding Aspects, wherein the third separated stream comprises 15 to 28 wt % C6+ hydrocarbons, based on a total weight of the third separated stream.
Aspect 12. The process of any one or more of the preceding Aspects, wherein the raffinate stream comprising C5 hydrocarbons is withdrawn from a side of the deisopentanizer assembly.
Aspect 13. The process of any one or more of the preceding Aspects, wherein passing the raffinate stream comprising C5 hydrocarbons to the reactor assembly to produce the reaction product stream comprises contacting the raffinate stream with a zeolite-based catalyst in the reactor assembly at a temperature of 100 to 210° C. and at a pressure of 10 to 30 barg (1,000 to 3,000 kilopascals) to produce the reaction product stream.
Aspect 14. The process of any one or more of the preceding Aspects, wherein the raffinate stream comprises pentene; and wherein the reactor assembly is configured and adapted to convert greater than or equal to 58 weight % of the pentene present in the raffinate stream to isoamylene, based on a total weight of the pentene in the raffinate stream.
Aspect 15. The process of any one or more of the preceding Aspects, wherein the raffinate stream comprises dienes or diolefins in an amount of less than or equal to 5 weight %, such as less than or equal to 1 weight %, based on a total weight of the raffinate stream.
Aspect 16. The process of any one or more of the preceding Aspects, wherein the raffinate stream comprises 0.10 to 1 wt % C6+ hydrocarbons, based on a total weight of the raffinate stream.
Aspect 17. The process of any one or more of the preceding Aspects, further comprising utilizing the second separated stream comprising isopentane to produce polyethylene or polystyrene.
Aspect 18. A system for producing isopentane and isoamylene comprising: a hydrodesulfurization assembly to produce a first stream from a feed stream comprising C5 hydrocarbons; a first separation assembly to separate the first stream and produce a second stream and a first separated stream; a deisopentanizer assembly to separate the second stream and produce a second separated stream comprising isopentane, a third separated stream comprising C6+ hydrocarbons, and a raffinate stream comprising C5 hydrocarbons; and a reactor assembly to produce a reaction product stream comprising isoamylene from the raffinate stream.
Aspect 19. The system of Aspect 18, further comprising a separation assembly to separate the reaction product stream and produce: (i) a recycle stream comprising C5 hydrocarbons, and (ii) a product stream comprising isoamylene; and wherein the recycle stream comprises isopentane in an amount of less than or equal to 10 weight %, based on a total weight of the recycle stream; wherein the product stream comprises 98 to 99.9 wt % isoamylene, based on a total weight of the product stream; and wherein the second separated stream comprises 68 to 75 wt % isopentane, based on a total weight of the second separated stream.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

Unless specified to the contrary herein, the total weight of each of the streams, feeds, feedstocks and outputs (e.g., feedstock stream; process streams; recycle streams; separated streams; product streams; raffinate streams, etc.) is 100 wt %.

As used herein, the term “C_{#} hydrocarbons” or “C_{#}” wherein “#” is a positive integer, describes hydrocarbons having # carbon atoms. Accordingly, the term “C₄ hydrocarbons” describes hydrocarbons having 4 carbon atoms. Moreover, the term “C_{#+} hydrocarbons” or “C_{#+}” describes hydrocarbons having # or more carbon atoms. Accordingly, the term “C₆₊ hydrocarbons” describes hydrocarbons having 6 or more carbon atoms (e.g., a mixture of hydrocarbons having 6 or more carbon atoms).

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

Although the processes, systems and methods of the present disclosure have been described with reference to exemplary embodiments thereof, the present disclosure is not limited to such exemplary embodiments and/or implementations. Rather, the processes, systems and methods of the present disclosure are susceptible to many implementations and applications, as will be readily apparent to persons skilled in the art from the disclosure hereof. The present disclosure expressly encompasses such modifications, enhancements and/or variations of the disclosed embodiments. Since many changes could be made in the above construction and many widely different embodiments of this disclosure could be made without departing from the scope thereof, it is intended that all matter contained in the drawings and specification shall be interpreted as illustrative and not in a limiting sense. Additional modifications, changes, and substitutions are intended in the foregoing disclosure. Accordingly, it is appropriate that the claims be construed broadly and in a manner consistent with the scope of the disclosure.

1. A process for producing isopentane and isoamylene comprising:
passing a feed stream comprising C5 hydrocarbons through a hydrodesulfurization assembly to produce a first stream;
passing the first stream to a first separation assembly to produce a second stream and a first separated stream;
passing the second stream to a deisopentanizer assembly to produce a second separated stream comprising isopentane, a third separated stream comprising C6+ hydrocarbons, and a raffinate stream comprising C5 hydrocarbons; and
passing the raffinate stream to a reactor assembly to produce a reaction product stream comprising isoamylene.

2. The process of claim 1 further comprising passing the reaction product stream to a separation assembly to produce: (i) a recycle stream comprising C5 hydrocarbons, and (ii) a product stream comprising isoamylene.

3. The process of claim 2 further comprising recycling the recycle stream to a cracker furnace assembly.

4. The process according to claim 2 or claim 3, wherein the recycle stream comprises isopentane in an amount of less than or equal to 16 weight %, such as less than or equal to 10 weight %, based on a total weight of the recycle stream.

5. The process according to claim 2, wherein the product stream comprises 98 to 99.9 wt % isoamylene, based on a total weight of the product stream.

6. The process of claim 1, wherein the second separated stream comprises 68 to 75 wt % isopentane, based on a total weight of the second separated stream.

7. The process of claim 1, wherein the deisopentanizer assembly is operated at 70 to 88 stages, and at a top temperature of 50 to 60° C. and at a bottom temperature of 70 to 85° C., and at a pressure of 2 to 3.1 barg (200 to 310 kilopascals).

8. The process of claim 1, further comprising forming the feed stream from a portion of an effluent from a cracker furnace assembly.

9. The process of claim 1, wherein the raffinate stream is heated via a heater assembly to a temperature of 70 to 160° C. prior to passing to the reactor assembly.

10. The process of claim 1, wherein prior to passing the feed stream comprising C5 hydrocarbons through the hydrodesulfurization assembly, the feed stream is hydrogenated; and
wherein the first separated stream comprises H₂S and C4 hydrocarbons.

11. The process of claim 1, wherein the third separated stream comprises 15 to 28 wt % C6+ hydrocarbons, based on a total weight of the third separated stream.

12. The process of claim 1, wherein the raffinate stream comprising C5 hydrocarbons is withdrawn from a side of the deisopentanizer assembly.

13. The process of claim 1, wherein passing the raffinate stream comprising C5 hydrocarbons to the reactor assembly to produce the reaction product stream comprises contacting the raffinate stream with a zeolite-based catalyst in the reactor assembly at a temperature of 100 to 210° C. and at a pressure of 10 to 30 barg (1,000 to 3,000 kilopascals) to produce the reaction product stream.

14. The process of claim 1, wherein the raffinate stream comprises pentene; and
wherein the reactor assembly is configured and adapted to convert greater than or equal to 58 weight % of the pentene present in the raffinate stream to isoamylene, based on a total weight of the pentene in the raffinate stream.

15. The process of claim 1, wherein the raffinate stream comprises dienes or diolefins in an amount of less than or equal to 5 weight %, such as less than or equal to 1 weight %, based on a total weight of the raffinate stream.

16. The process of claim 2, wherein the raffinate stream comprises dienes or diolefins in an amount of less than or equal to 5 weight %, such as less than or equal to 1 weight %, based on a total weight of the raffinate stream.

17. The process of claim 3, wherein the raffinate stream comprises dienes or diolefins in an amount of less than or equal to 5 weight %, such as less than or equal to 1 weight %, based on a total weight of the raffinate stream.

18. The process of claim 4, wherein the raffinate stream comprises dienes or diolefins in an amount of less than or equal to 5 weight %, such as less than or equal to 1 weight %, based on a total weight of the raffinate stream.

19. The process of claim 5, wherein the raffinate stream comprises dienes or diolefins in an amount of less than or equal to 5 weight %, such as less than or equal to 1 weight %, based on a total weight of the raffinate stream.

20. The process of claim 6, wherein the raffinate stream comprises dienes or diolefins in an amount of less than or equal to 5 weight %, such as less than or equal to 1 weight %, based on a total weight of the raffinate stream.
